(19) United States
(12) Patent Application Publication
(10) Pub. No.: US 2009/0081149 A1
(43) Pub. Date: Mar. 26, 2009

(54) Skin And Hair Care

(76) Inventors: Stephen Watkins, Kent (GB); Severine Lieurey, Kent (GB)

Correspondence Address: MORGAN LEWIS & BOCKIUS LLP, 1111 PENNSYLVANIA AVENUE NW, WASHINGTON, DC, 20004, US

(21) Appl. No.: 11/988,023

(22) PCT Filed: Jun. 27, 2006
(86) PCT No.: PCT/GB2006/002359

§ 371 Date: Dec. 28, 2007

(30) Foreign Application Priority Data
Jun. 29, 2005 (GB) 0513193.3

(51) Int. Cl. A61K 8/97; A61Q 5/12; A61Q 19/00

(52) U.S. Cl. 424/74; 424/115; 514/23

(57) ABSTRACT

A fermentation product of soya bean extract, e.g. soya milk, provides conditioning benefits for skin, particularly moisturising benefits, namely increased moisture, and conditioning benefits for hair, particularly reduction of fly away behaviour and improved curl retention.

### FIELD OF THE INVENTION

This invention concerns improvements in skin or hair care.

### BACKGROUND TO THE INVENTION

Milk and dairy products have been used in cosmetic skin care applications for many hundreds of years, as a way to add moisture and fats to the skin in order to condition and moisturise the skin. Fermented milk (yoghurt) has also been used historically for skincare applications, again to add moisture and fats to the skin but in a more viscous format.

JP 2003-113066 discloses an anti-wrinkle cosmetic composition comprising vitamin C or a derivative thereof and a soy bean extract lactic fermentation product. Vitamin C and derivatives thereof are known to have potential anti-wrinkle benefits, but practical difficulties arise in producing stable formulations. The fermentation product was included with the aim of improving stability.

The present invention is based on the surprising finding that a fermentation product of soya bean extract, e.g. soya milk, provides conditioning benefits for skin, particularly moisturising benefits, namely increased moisture, and conditioning benefits for hair, particularly reduction of fly away behaviour and improved curl retention.

### SUMMARY OF THE INVENTION

In one aspect the invention provides use of a fermentation product of soya bean extract for providing conditioning benefits for skin or hair.

The invention also provides use of a fermentation product of soya bean extract as an ingredient in a composition for treatment or care of skin or hair for providing conditioning benefits for skin or hair.

Also included within the scope of the invention is a method of providing conditioning benefits to skin or hair, comprising topical application to skin or hair of a fermentation product of soya bean extract.

In a further aspect, the invention provides a composition, particularly a cosmetic composition, for the treatment or care of skin, comprising a fermentation product of soya bean extract.

The invention also provides a composition, particularly a cosmetic composition, for the treatment or care of hair, comprising a fermentation product of soya bean extract.

Tests by the inventors have shown that a fermentation product of soya bean extract, when topically applied, provides conditioning benefits for skin, particularly skin moisturising benefits in the form of increased skin moisture, and conditioning benefits for hair, including styling benefits such as reduced fly away and improved curl retention. The hair conditioning benefits are thought to be related to the protein content of the fermentation product, while the skin conditioning benefits are thought to be related to the sugar and fat content. Skin conditioning benefits are different from anti-wrinkle effects.

The soya bean extract conveniently comprises an aqueous soya extract, which is commonly referred to as soya milk (also known as “Tonyu”), despite the fact that the material is not related to animal-derived milk. Soya milk can be obtained by washing and crushing de-skinned soya beans with water, followed by filtering to remove the solids, leaving the soya milk.

The fermentation product is desirably a product of lactic acid fermentation. This is desirably achieved using aerobic bacteria, particularly using a Lactobacillus species and a Streptococcus species, e.g. Lactobacillus bulgaricus and Streptococcus thermophilus. Suitable fermentation techniques are well known to those skilled in the art.

The fermentation product is conveniently fermented aqueous soya extract (fermented soya milk), which is commonly referred to as soya yoghurt. Soya yoghurt does not relate to animal-derived yoghurt, and is made by fermentation of soya milk. Soya yoghurt is commercially available, e.g. from Sojasun.

The fermented product is preferably heat treated to destroy the bacteria. Suitable heat treatment methods are well known to those skilled in the art, e.g. treating at a temperature in excess of 100° C. for less than 1 minute.

The fermentation product is desirably used in dried condition for inclusion in a composition (e.g. in the form of a powder or granules) rather than in liquid condition. Use of a dried fermentation product compared to one in liquid condition as an ingredient in a skin or hair care or treatment composition has certain practical benefits, in particular in terms of a longer shelf life of the dried ingredient, lower content of microorganisms and avoidance of risk of microbial growth. Material in dry condition can also be incorporated into powdered or anhydrous products.

The fermentation product e.g. soya yoghurt is thus desirably dried, e.g. spray dried, to produce a dry powder or granules. Suitable drying techniques are well known to those skilled in the art.

The invention thus also provides a method of making a composition for care or treatment of skin or hair, comprising including as an ingredient a fermentation product of a soya bean extract in dried condition.

The fermentation product, particularly when in dried condition, preferably has a low or zero fat content, e.g. having a lipid content of less than 10% by weight, preferably less than 5% by weight, more preferably less than 3% by weight. Low fat levels can be achieved by e.g. solvent extraction of the product with ethanol prior to spray drying. For example, a typical commercially available natural soya yoghurt contains approximately 2.5% by weight lipids with a solids content of about 10% by weight. Spray drying produces a powder wherein the fermentation product component has a natural lipids content of about 25% by weight. The lipids can be reduced by solvent extraction with ethanol prior to spray drying, resulting in a spray dried powder with a lipids content of less than 10% by weight, possibly less than 5% by weight, or less than 3% by weight. Alternatively, the lipid content of the dried fermentation product can be reduced by the addition of other components, such as flow aids, resulting in a dried product with a lipids content of less than 25% by weight, possibly less than 15% by weight, or less than 10% by weight.

The invention thus includes within its scope a dried fermentation product of soya bean extract with a lipids content of less than 10% by weight, preferably less than 5% by weight, more preferably less than 3% by weight.

The fermentation product desirably has a low or zero sugar content, e.g. having a sugar content of less than 10% by weight. A typical commercially available natural soya yoghurt contains about 2.5% by weight sugars with a solids content of about 10% by weight. The sugars are naturally present in the soy bean milk prior to fermentation. Additional sugar may be added to improve the fermentation process or for taste reasons. Spray drying produces a powder wherein the fermentation product component has a natural sugars content of about 25% by weight. The soya content of the dried fermentation product can be reduced by the addition of other components such as flow aids, resulting in a dried product with a sugar content of less than 25% by weight, possibly less than 15% by weight, or less than 10% by weight.

One or more flow aids are conveniently added to the fermentation product, e.g. soya yoghurt, prior to drying to assist in production of a free-flowing, non-sticky powder. Suitable flow aids are well known in the art and include mannitol, maltodextrin, lactose or similar sugar or polyol. The resulting dried product could contain an amount of flow aid in the range 5 to 75% by weight. It has also been found that the addition of soy proteins and/or hydrolysed soy proteins prior to drying assists in the production of a free-flowing powder.

The resulting dried product could contain from 2 to 25% by weight of hydrolysed soy proteins.

Addition of flow aids is a convenient way of reducing the fat and/or soya content of the dried fermentation product.

In a preferred embodiment the fermentation product is spray dried and comprises 30% by weight fermented soya extract, 11% by weight hydrolysed soy protein and 59% by weight maltodextrin.

One or more preservatives are conveniently included in the fermentation product, to prevent degradation. The preservative may be, e.g., an anti-oxidant or an agent to prevent microbial growth. Preservative is typically present at low levels, e.g. in the range 0.01 to 2% by weight of a skin or hair care or treatment composition. Examples of suitable anti-oxidants include, but are not limited to, tochopherol, rosemarinic acid, polyphenolic compounds, butylated hydroxy toluene (BHT), butylated hydroxy anisole (BHA). Examples of suitable anti-microbial agents include, but are not limited to, paraben, benzoate (or acid), sorbate. The preservatives can be added at any time during processing but are preferably added following the fermentation process and prior to spray drying.

Use of a flow aid and/or preservative can assist in forming a stable dried powder, useful as an ingredient in skin and hair care compositions.

The invention is applicable to a wide range of compositions, including shampoos, hair conditioners (wash-off and leave-in), hair styling products (gel, spray, cream, mousse), hair treatment products (hair colouring products, perms and bleaches), skin care products (creams, gels, lotions, sprays, sticks, mousse), colour cosmetics (pressed and free powders, sticks, liquids, gels), suncare/aftersun products (creams, gels, lotions, sprays, sticks, mousse), skin treatment products (anti-acne, anti-cellulite, anti-irritant, anti-aging, firing), fragrance products (creams, gels, lotions, sprays, sticks, mousse), bath and shower products, soaps and hand washes, etc.

Such compositions can be otherwise of generally conventional formulation, as is well known to those skilled in the art.

Typical ingredients included in such compositions include the following:

Aqua [water], alkanolamide surfactants, alkyl polyglucose derivatives, alkyl ether sulphate surfactants, allantoin, Aloe barbadensis Mil (Aloe Vera Linne), alpha-hydroxycarboxylic acids, aluminium chlorhydrate and its derivatives, aluminium salts, amide derivatives, amino acids, amphoteric surfactants, ascorbic acid and its salts, bentonite and hectorite clays, benzyl alcohol, bisabolol, butane propellants, carboxy vinyl polymer water soluble salts, carboxy methylcellulose, carrageenan, cetrimonium salts, cholesterol, cocamidopropyl betaine, depilatories, dihydroxyacetone, dimethyl ether, ethanol, fatty acids, fatty acid citrate esters, fatty alcohols, fragrance ingredients, glucose esters of higher fatty acids, glycerin, glyceryl stearates, guar gum, gum arabic, h₂o, hyaluronic acid, hydroxybenzoic acids, hydroquinone, isopropyl alcohol, kojic acid, lanolin, lauryl betaine, lipids extracted from the biomass of microorganisms, yeasts, moulds and bacteria, liposomes, liquorice (Glycyrrhiza glabra) and its components (glycyrrhetinic acid, glycyrrhizic acid, etc.), locust bean gum, low molecular weight acidic mucopolysaccharides & their salts, low molecular weight humectant components, magnesium aluminium silicate, methacrylate polymers, mineral oils, mineral powders, natural or synthetic amino acid with protein or peptide bond, natural moisturising factor (NMF) ingredients e.g. pyrrolidone carboxylic acid, non-volatile silicones, oil agent(s), oil matter, oligosaccharide(s), organic acids, pantothenic acid and its derivatives, petroleum jelly, phospholipid, polysaccharide, polyvinyl alcohol, polypeptides, proteins, piroctone olamine, quaternised silicone derivatives, raffinose, saponins, sodium chloride, sodium cocoyl isethionate, sodium hyaluronate, sources of linoleic acid, sterols, sterol esters, sucrose, sugar esters of higher fatty acids, sunscreens, surfactants, talc, tea tree oil (Melaleuca spp.), thioglycolic acid and its derivatives, titanium dioxide, tocopherol, triclosan, mono- or di- or triglycerides, vegetable oils, vitamins and analogues, volatile silicone fluids, water soluble moisture-retaining agents, water soluble polymers, waxes, zinc oxide, zinc pyrithione, etc.

Skin care compositions in accordance with the invention do not rely on the presence of materials such as vitamin C (L-ascorbic acid) or derivatives thereof to give skin conditioning benefits, particularly moisturising benefits in the form of increased moisture control of skin. Certain embodiments of compositions for the treatment or care of skin in accordance with the invention thus need not and do not include vitamin C or derivatives thereof.

A fermentation product of soya bean extract has low allergenic properties and so is acceptable for use on sensitive and susceptible individuals.

Skin and hair care compositions in accordance with the invention typically include the fermentation product in an amount of less than 5% by weight, more typically less than 2% by weight. Use of the fermentation product of at a level of 1% by weight or below will give beneficial effects in most product types.

The invention will be further described, by way of illustration, in the following Examples and with reference to the accompanying drawings, in which:

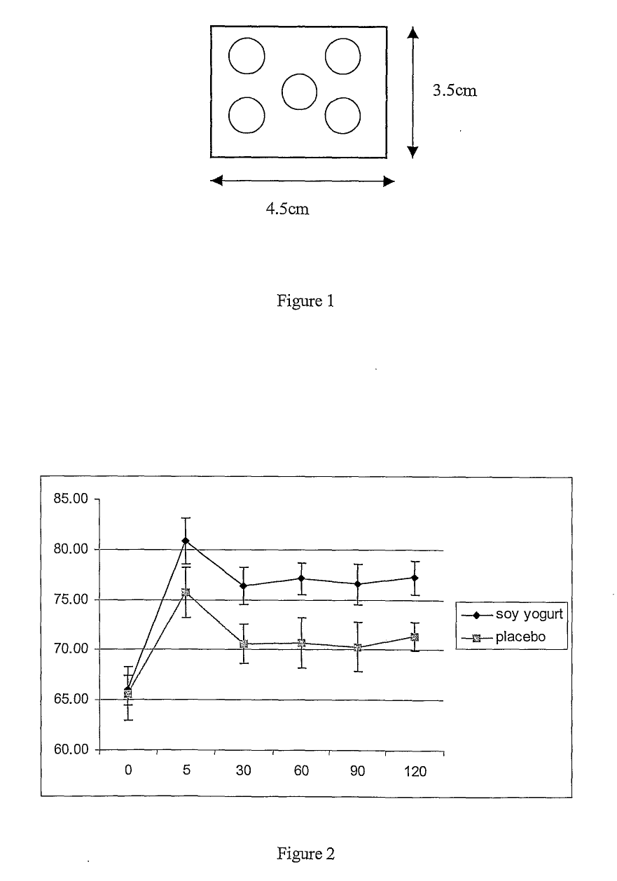

FIG. 1 is a schematic diagram of the pattern of test regions used for measuring moisture content of skin in the tests of Example 1;

FIG. 2 is a graph of capacitance (in arbitrary units) versus time (in minutes) showing the results of Example 1;

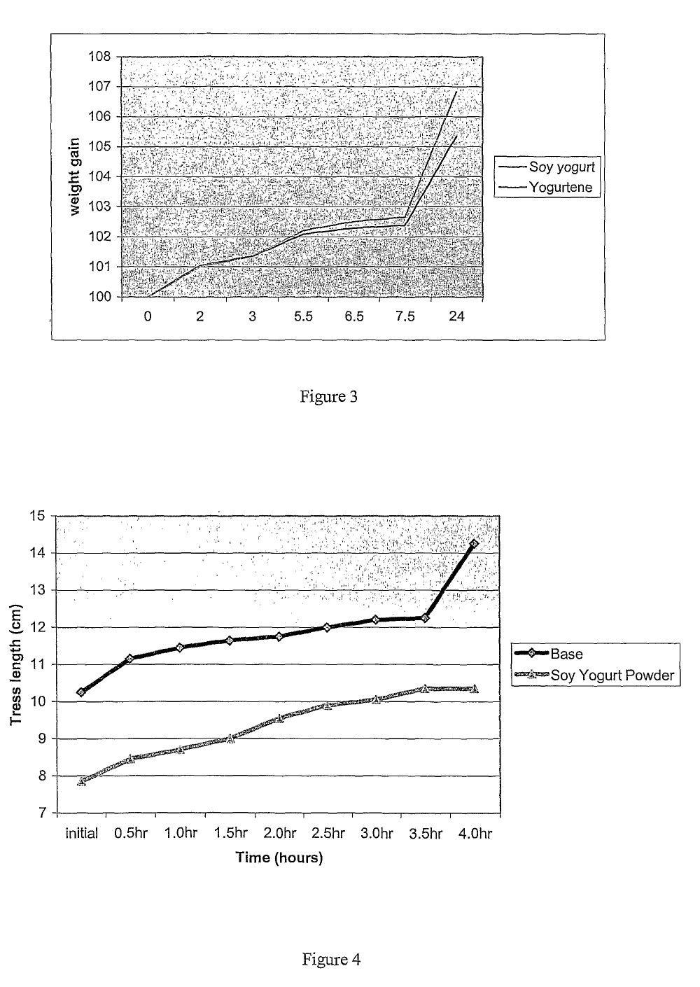

FIG. 3 is a graph of weight gain versus time (in hours) showing the results of Example 2;

FIG. 4 is a graph of tress length (in cm) versus time (in hours) showing the results of Example 3; and

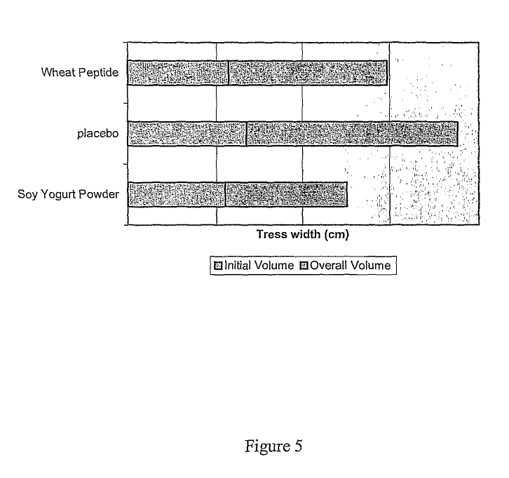

FIG. 5 is a chart of tress width (in cm) showing the results of Example 4.

The experiments below were carried out using a dried soya yoghurt powder, prepared as follows. Sojasum soya yoghurt with a solids content of 10% by weight was heat treated to 100° C. for less than a minute to destroy any bacteria. The heat treated soya yoghurt was then spray dried. Spray drying was carried out by pumping the fermented soy through a heated spray nozzle. The nozzle temperature was set to 200° C. to cause flash evaporation of the water. The resulting powder contained approximately 50% by weight protein, approximately 25% by weight sugars and approximately 25% by weight lipids. The powder used contained no flow aids or preservatives.

### EXAMPLE 1

Experiments were carried out to investigate the effect of spray dried soya yoghurt powder on skin moisture, in particular to see whether the powder incorporated into a standard skin care formulation can improve the moisture content of skin.

Skin moisture is measured using electric skin properties. The methods are based on the conductance, impedance and capacitance of the stratum corneum. Capacitance is the established standard method (e.g. corneometry). The principle of measurement is based on the dielectric constant of water, which strongly differs from that of other substances. The capacitor's electric field, influenced by the scatter field in the moist stratum corneum, is altered proportionally to the moisture content. The higher the measured capacitance value the higher the moisture content.

A square (3.5 cm×4.5 cm) was drawn with make-up pen on the right and left arm of each of 9 volunteers. The water content of the skin was measured on five different places of square, in a pattern as shown in FIG. 1. The average and standard deviation was then calculated.

The skin water content was measured initially before product application.

A drop of cream (approx 0.03 g), with and without soya yoghurt powder, was spread evenly on each square. Formulation details of the creams are given below.

Skin capacitance, indicative of the water content of the skin, was measured after 5 minutes and then every 30 minutes after application for a total of 120 minutes. The results, with capacitance measured in arbitrary units, are set out in Table 1 below and are shown graphically in FIG. 2.

TABLE 1

| | Time (mins) | | | | | |
|---|---|---|---|---|---|---|
| | 0 | 5 | 30 | 60 | 90 | 120 |
| Cream (soya yogurt powder) | 65.96 | 80.89 | 76.36 | 77.09 | 76.56 | 77.23 |
| Standard deviation | 1.47 | 2.29 | 1.89 | 1.60 | 2.02 | 1.68 |
| Cream (placebo) | 65.58 | 75.73 | 70.58 | 70.69 | 70.31 | 71.38 |
| Standard deviation | 2.68 | 2.52 | 1.99 | 2.51 | 2.45 | 1.45 |

The test was carried on 9 volunteers. The cream containing the soya yogurt powder was shown to increase the level of moisture in the skin significantly as compared with the placebo cream without soya yoghurt powder.

### Skin Cream Formulation

| | | Soya Yogurt | Placebo |
|---|---|---|---|
| | | Cream | Cream |
| | | wt % | wt % |
| Phase A | | | |
| ESTOL 3609 | Triethylhexanoin | 5.00 | 5.00 |
| DC 245 Fluid | Cyclopentasiloxane | 2.00 | 5.00 |
| ARLAMOL E | PPG-15 Stearyl Ether | 2.00 | 2.00 |
| Laurex CS | Cetearyl Alcohol | 2.00 | 2.00 |
| Phase B | | | |
| PRICERINE 9091 | Glycerine | 2.00 | 2.00 |
| ARLATONE V-175 | Sucrose palmitate, | 1.10 | 1.00 |
| | glyceryl stearate, | | |
| | glyceryl stearate citrate, | | |
| | sucrose, | | |
| | mannan and xanthan | | |
| | gum | | |
| Water | Aqua | To 100 | To 100 |
| Veegum Ultra | Magnesium Aluminium | 0.10 | 0.10 |
| | Silicate | | |
| Soya yoghurt | | 0.50 | 0.00 |
| powder | | | |
| Phase C | | | |
| Phenonip | Phenoxyethanol, | 1.00 | 1.00 |
| | methylparaben, | | |
| | ethylparaben, butylparaben, | | |
| | propylparaben and | | |
| | isobutylparaben | | |

The creams were made as follows.

Phase A was made by mixing all the ingredients and heating to 80° C.

Phase B was made by adding the water to a beaker, add the soya yoghurt powder, veegum ultra, Pricerine 9091 and Arlatone V-175, stirring well between each addition, with heating to 80° C.

Phase A was poured into phase B with high shear mixing. The mixture was cooled to 40° C. The ingredients of phase C were added by stirring.

### EXAMPLE 2

### Moisture Uptake of Soya Yoghurt Powder and Dairy Yoghurt Powder

The aim of this test was to measure the water absorbing properties of soya yoghurt powder versus Yogurtene dairy yoghurt powder when left open in a high humidity cabinet. Yogurtene is available from Quest International. Yogurtene is a Trade Mark.

### Protocol and Results

2 open jars containing soya yoghurt powder or dairy yoghurt powder were left open in oven maintained at 37° C. and 70% humidity for a period of 24 hours. The weight of each jar was monitored over this time as a direct measure of water uptake.

The weight gain was calculated as set out below

Weight gain=(weight−T/initial weight−T)×100

T=Jar weight

The results are shown in Table 2 below and in FIG. 3.

TABLE 2

| | Time (hours) | | | | | | |
|---|---|---|---|---|---|---|---|
| Product | 0 | 2 | 3 | 5.5 | 6.5 | 7.5 | 24 |
| Soya Yoghurt | 100.00 | 101.04 | 101.35 | 102.08 | 102.29 | 102.37 | 105.36 |
| Dairy Yoghurt | 100.00 | 101.01 | 101.35 | 102.20 | 102.51 | 102.65 | 106.83 |

The results show that the soya yoghurt powder seems to be slightly less hydroscopic than the dairy yoghurt powder after 24 hours but still takes up a considerable amount of water (approx. 70%).

### EXAMPLE 3

### Curl Retention of Soya Yoghurt Powder

### Pre-Treatment

Hair tresses, 23 cm in length, were soaked overnight in deionised water. The tresses were then dipped into a 10% surfactant solution (details of which are given below) and again rinsed with deionised water. Each tress was then dried.

The surfactant (before dilution in water to give a 10% w/w solution) had the following composition:

| | |
|---|---|
| Water | 82% w/w |
| Sodium laureth (2) sulphate (70% solution) | 12% w/w |
| Cocamidopropyl betaine (30% solution) | 5% w/w |
| Citric acid | q.s. |
| Sodium chloride | q.s. |
| Antimicrobial Preservative | q.s. |

### Test

A solution of 1% w/w soy yoghurt powder in deionised water was prepared. This was compared to deionised water (base). Tresses were soaked in either the water (base) or the soya yoghurt powder solution, for one hour. After this period the tresses were rinsed in deionised water, curled onto small/medium perm rods and dried using a Braun 1250 Silencio hairdryer (Braun and Silencio are Trade Marks), at a distance of 10 cm, until the hair was dry. Once dried, the hair was attached to a retort stand for support and removed from the rods. Each tress was measured at its initial unwound length and monitored at half hourly intervals.

The results are shown in the graph of FIG. 4, representing the curl retention of each product.

The graph of FIG. 4 identifies the initial and overall drop length of each tress. The percentage curl retention is a function of time and calculated using the following equation:

$$\% Curl retention = \frac{\begin{matrix}length of fully extended hair - \\ length hair after time\end{matrix}}{\begin{matrix}length of fully extended hair - \\ length of hair after curling\end{matrix}}$$

% curl retention values are as follows:

| | % Curl Retention |
|---|---|
| Base | 68.6% |
| Yoghurt | 85.4% |

This test shows that the soya yoghurt powder helps to maintain the curl, providing improved hair curl retention and styling benefits.

### EXAMPLE 4

### Fly-Away

Tests were carried out to investigate the fly-away effects of the soya yoghurt powder compared with those for wheat peptide (Naturein Wheat Peptide, available from Quest International) (Naturein is a Trade Mark) and placebo (water).

Hair tresses, 23 cm in length, were washed in a 10% surfactant solution (as used in Example 3) and rinsed with deionised water before being dried. Separate 1% aqueous solutions of soya yoghurt powder and wheat peptide were prepared. Tresses were soaked in the soya yoghurt powder solution or the wheat peptide solution or deionised water (placebo) for 1 hour. After this time, the tresses were rinsed with deionised water and dried using a hair drier as in Example 3. The tresses were then placed on a flat surface and the width of the tress measured. Each tress was combed 10 times with even downward strokes and then the widths measured and compared with measurements of tress width taken before treatment. The results are shown in FIG. 5.

A lower tress width indicates that the hair has more ‘body’ and is less able to fly-away due to static charge.

The results obtained indicate that the addition of 1% Soy yoghurt powder in a rinse off formulation will provide styling benefits to hair, increasing both the holding of a style and preventing fly-away.

Typical formulations of compositions in accordance with the invention include the following. All of the compositions include dried soya yoghurt powder, prepared by spray drying Sojasun soya yoghurt as described previously.

### EXAMPLE 5

### Shampoo

| Trade Name | INCI Name | % w/w |
|---|---|---|
| Deionised water | | to 100.00 |
| Texapon NS/IOS | Sodium Laureth Sulfate (28%) | 35.00 |
| Tego Betain L7 | Cocamidopropyl Betaine | 10.00 |
| Comperlan COD | Cocamide DEA | 2.00 |
| NaCl | | 2.10 |
| Euperlan PK771 | Glycol Distearate & Sodium | 1.00 |
| | Laureth Sulfate & | |
| | Cocamide MEA & Laureth-10 | |
| Soya Yogurt Powder | | 0.50 |
| Suttocide A | Sodium Hydroxymethylglycinate | 0.10 |
| QUEST PERFUME | | q.s. |

### Method:

Pre-mix water, Cocamidopropyl Betaine and Sodium Laureth Sulphate under propeller stirrer. Once mixed thoroughly add Cocamide DEA, Soya yogurt Powder and Euperlan. Finally add Suttocide A and add fragrance as required.

### EXAMPLE 6

### Aerosol Dry Shampoo

This product should be sprayed into the hair and then removed by brushing until no powder is visible. It contains oil-absorbing starch, plus Whey peptide for hair benefits.

| Trade Name | INCI Name | % w/w |
|---|---|---|
| Powder concentrate: | | |
| Tapioca Pure | Tapioca Starch | 39.80 |
| Talc | | 21.00 |
| QUESTICE PLUS | Menthyl PCA & Menthol & DPG | 12.00 |
| Soya Yogurt Powder | | 10.00 |
| Dry Flo Elite LL | Aluminium Starch | 9.00 |
| | Octenylsuccinate & Lauroyl | |
| | Lysine | |
| Estol IPP | Isopropyl Palmitate | 2.00 |
| Nipagin M | Methylparaben | 0.20 |
| QUEST FRAGRANCE | | q.s. |
| Aerosol fill | Powder concentrate | 12.00 |
| | Hydrocarbon Propellant (40 psig) | 88.00 |

### Method:

Mix the IPP and perfume with the Questice Plus, add very gradually to the combined powders, and mix well using an electric blender. Fill the aerosol cans. (NB. A valve suitable for use with powder products must be used).

### EXAMPLE 7

### Scoopable Bath Yoghurt

A rich bath or shower product, with the texture and benefits of natural yoghurt. Apply to the body using the hands or a sponge, for a gently moisturizing wash

| Trade Name | INCI Name | % w/w |
|---|---|---|
| Deionised water | | to |
| | | 100.00 |
| Texapon nso/is | Sodium Laureth Sulfate | 53.5 |
| Empigen bb | Lauryl Betaine | 8.0 |
| Protamate 6000ds | PEG-150 Distearate | 2.0 |
| OPACIFIER E308 | Sodium Styrene Acrylates Divinylbenzene | 1.0 |
| | Copolymer | |
| Ceramide II | | 0.05 |
| Nervanaid ba2 | Disodium EDTA | 0.1 |
| Germaben ii | Diazolidinyl Urea & Propylene Glycol & | 0.3 |
| | Methylparaben & Propylparaben | |
| Soya yogurt Powder | | 0.5 |
| QUEST PERFUME | | q.s |

### Method:

Combine the water, Nervanaid and Protamate, and heat to 75° C. until clear and uniform. Cool to 50° C. and stir in the soya yogurt powder. Separately combine the Empicol and Questamide H, and warm gently with stirring until fully dissolved. Add to the main batch, stirring to incorporate. Add the Opacifier, Germaben and perfume, stirring until uniform. Finally, add the Empigen, stirring slowly until homogeneous.

### EXAMPLE 8

### Hair Conditioner

A light emulsion that develops pearlescence over time. Contains milk vitamins and lipids to nourish the hair and scalp, for added strength and shine.

### Formulation:

| Trade Name | INCI Name | % w/w |
|---|---|---|
| Deionised water | | to 100.00 |
| Cetyl Alcohol | | 4.00 |
| Schercomid CME | Cocamide MEA | 2.00 |
| Lexamine S13 | Stearamidopropyl Dimethylamine | 2.00 |
| Dehyquart A | Cetrimonium Chloride | 1.50 |
| Soya Yogurt | | 1.00 |
| Powder | | |
| Lactic Acid 85% | | 1.00 |
| Sodium Stearate | | 0.90 |
| D-Panthenol 75L | Panthenol | 0.75 |
| Gennaben II | Propylene Glycol & Diazolidinyl Urea & | 0.30 |
| | Methylparaben & Propylparaben | |
| Apricot Extract | Prunus Armeniaca (Apricot) Fruit Extract | 0.25 |
| QUEST | | q.s. |
| PERFUME | | |

### Method:

Combine the water, soya yoghurt powder, Dehyquart and Lactic Acid, and heat to 80° C. Separately combine the Cetyl Alcohol, Schercomid, Lexamine, Sodium Stearate, and heat to 80° C. Add to the water phase, and homogenise until smooth. Cool to room temperature, and add the remaining ingredients.

### EXAMPLE 9

### Leave-on Conditioner for Hair

| Trade Name | INCI Name | % W/W |
|---|---|---|
| Phase A | | |
| Deionised Water | Aqua | 84.50 |
| Soya Yogurt Powder | | 0.50 |
| Down Corning 1401 | Cyclomethicone (and) Dimethiconol | 8.00 |
| Fluid | | |
| Phase B | | |
| Propylene glycol | | 2.80 |
| Sepigel 305 | Polyacrylamide, C13-14 Isoparaffin & | 3.50 |
| | Laureth-7 | |
| Sinodor | Citronellyl Methylcrotonate | 0.50 |

### Method:

### Phase A

Mix the Propylene Glycol with DC1401 until clear.

### Phase B

Add 0.5% of the Sepigel 305 to the water, mix with high shearing until homogeneous.

Add phase A to phase B, using a Silverson mixer until homogeneous.

Add the remaining Sepigel and manually stir until smooth and thick.

### EXAMPLE 10

### Hair Moisturising Milk

This opaque product contains a blend of conditioning agents to improve the shine, body, and manageability of the hair. It will not build-up on the hair, to cause oiliness or limpness, and so is suitable for frequent use.

| FORMULATION: | | % w/w |
|---|---|---|
| LAUREX CS | Cetearyl Alcohol | 0.90 |
| AMMONYX 4A | Stearalkonium Chloride | 2.80 |
| NATROSOL 250HHR | Hydroxyethyl Cellulose | 0.70 |
| YOGURTENE | Yogurt Powder | 0.50 |
| CITRIC ACID | Citric Acid | 0.30 |
| MERQUAT PLUS 3330 | Polyquaternium-39 | 2.00 |
| d-PANTHENOL 75L | Panthenol | 1.00 |
| GERMABEN II-E | Propylene Glycol & Diazolidinly | 0.30 |
| | Urea & | |
| | Methylparaben & Propylparaben | |
| PURIFIED WATER | | to 100.00 |
| QUEST PERFUME | Parfum | q.s. |
| Soya Yogurt Powder | | 0.30 |

### Method:

Disperse the soya yogurt powder and them the Natrosol in water with propellor stirring, heat to wet out. Add the Yogurtene, Merquat, citric acid, and Panthenol, and heat to 70° C. Melt the Ammonyx and Laurex CS, then add to the water phase. Propellor stir to mix thoroughly (homogeniser can be used for large batches), then continue to scrape stir until cool. Add the Germaben and perfume. Mix well, avoiding entrainment of air. Finally, add the dye solution.

### EXAMPLE 11

### Styling Hair Foam (Without Aerosol & Alcohol)

| | | INCI | % w/w |
|---|---|---|---|
| A. | Amaze (1) | (Corn Starch Modified) | 1.00 |
| | Soya Yogurt powder | | 0.70 |
| | Water | (Aqua) | 50.00 |
| B. | Water up to | (Aqua) | 100.00 |
| | Alpantha (2) | (Panthenol, Allantoin) | 1.00 |
| C. | Brij 700 (3) | (Steareth-100) | 1.00 |
| | Manroteric CAB (4) | (Cocamidopropyl Betaine) | 0.50 |
| D. | Kemaben 2 (2) | (Propylene Glycol, Diazolidinyl | 1.00 |
| | | Urea, Methylparaben, | |
| | | Propylparaben) | |

### Manufacture:

Dissolve Amaze in water stirring. Heat phase A at 65° C. Add the phases B and C. Stir at 65° C. for 30 minutes. Allow to cool down at 40-35° C. stirring. Add phase D and cool down at room temperature stirring slowly.

pH=6.21

### Suppliers:

(1) National Starch (Quimassó)

### (2) Akema

(3) Uniqema (Massó & Quimassó)
(4) TensaChem (Quimassó)

### EXAMPLE 12

### Natural Hair Styling Soft Gel

| | | INCI-Name | % w/w |
|---|---|---|---|
| A. | Jaguar HP 105 (1) | (Hydroxypropyl Guar) | 1.20 |
| | Amaze (5) | (Corn Starch Modified) | 1.20 |
| | Soya yogurt powder | | 1.50 |
| | Water up to | (Aqua) | 100.00 |
| | Benzophenone-4 | (Benzophenone-4) | 0.10 |
| | Alpantha (4) | (Panthenol, Allantoin) | 1.00 |
| B. | Kemaben 2 (5) | (Propylene Glycol, Diazolidinyl | 1.00 |
| | | Urea, Methylparaben, | |
| | | Propylparaben) | |
| C. | Tween 20 (4) | (Polysorbate 20) | 0.50 |
| | Perfume Morning | (Parfum) | q.s. |
| | 870601/18 (6) | | |
| | Alcohol denat. | (Alcohol denat.) | 15.00 |

### Manufacture:

Dissolve Amaze with 50% of water at room temperature for 20 min. approx. Add Alpantha, benzophenone-4 and Naturein Wheat Protein till get an homogeneous solution. Dissolve Jaguar HP 105 with other part of water stirring moderately and heating a little (40° C.). Mix till gel is well formed. Mix the two parts of water whilst stirring slowly. Add phase B. Mix well ingredients of phase C and add to AB.

Viscosity (21° C.): 40,800 mPa·s (Brookfield LVT, Spindle 7, 1.5 rpm)
pH=5.29 (citric acid, 10%)

### Suppliers:

(1) Rhône-Poulenc
(2) National Starch (Quimassó)
(3) Quest C.I. (Massó & Quimassó)
(4) Uniqema (Massó & Quimassó)

### (5) Akema

### (6) Chemir

### EXAMPLE 13

### Hair Sculpting Lotion

| Trade Name | INCI Name | % w/w |
|---|---|---|
| Phase A | | |
| Purified water | | 50.00 |
| Natrosol 250 hhr | Hydroxyethyl cellulose | 0.50 |
| Phase B | | |
| Purified water | | to 100.00 |
| Gafquat hs-100 | | 10.00 |
| Panthequat | Steardimonium panthenol. | 1.25 |
| Soya Yogurt Powder | | 0.10 |
| Preservative | | as required |
| Phase c | | |
| Eumulgin 1 | PPG-CETEARETH-9 | 2.50 |
| Quest fragrance | | as required |

PREPARATION: Disperse the Natrosol in the water of phase A, with rapid stirring. Combine the ingredients of phase B and add to phase A. Premix the ingredients of phase C, and add to the bulk with stirring until homogenous.

### EXAMPLE 14

### Bleaching Cream (Hydrogen Peroxide Based)

This bleaching cream based on hydrogen peroxide is simple and cheap to manufacture.

| Trade name | % w/w |
|---|---|
| Phase A: | |
| Solan E (PEG 75 lanolin) | 5.00 |
| Cetyl alcohol | 10.00 |
| Phase B: | |
| Hydrogen peroxide (100 | 6.00 |
| vol) | |
| Soya yogurt powder | 0.10 |
| Purified water | To 100.00 |
| Preservative | As required |
| Phase C: | |
| Quest fragrance | As required |

PREPARATION: Heat phase A to 50° C., Warm phase B. Using an overhead stirrer add phase A to B and continue to stir until cool. When cool add the fragrance.

### EXAMPLE 15

### Oil in Water Hair Mascara (for Fine Hair)

| | | INCI-Name | % w/w |
|---|---|---|---|
| A. | Brij 35 (1) | (Laureth-23) | 1.50 |
| | Ecorol 68/30 f (2) | (Cetearyl Alcohol) | 6.00 |
| | Arlamol HD (1) | (Isohexadecane) | 1.00 |
| B. | Water up to | (Aqua) | 100.00 |
| | Empigen BCN 83 (3) | (Stearalkonium | 0.50 |
| | | Chloride) | |
| | Pricerine 9091 (1) | (Glycerin) | 2.00 |
| | EDTA Na2 | (Disodium EDTA) | 0.10 |
| C. | Soya Yogurt powder | | 2.00 |
| D. | Kemaben 2 (5) | (Propylene Glycol, | 1.00 |
| | | Diazolidinyl Urea, | |
| | | Methylparaben, | |
| | | Propylparaben) | |
| E. | Purac HiPure 90 (6) | (Lactic Acid) | pH = 4.0-4.5 |
| F. | Perfume Frescura 3467 (7) | (Parfum) | q.s. |

### Manufacture:

Heat phases. A and B to 75° C. Add phase B to A stirring moderately. Homogenise AB for 1½ minutes (Silverson: 3.000 rpm). Allow to cool down to 40° C. stirring slowly. Add phases C and D. Adjust final pH with phase E. Add perfume. Cool down to room temperature stirring slowly.

Viscosity: 43.680 mPa·s (Brookfield LVT, Spindle F, 6 rpm)

### Suppliers:

(1) Uniqema (Massó & Quimassó)
(2) Ecogreen (Massó)

### (3) Huntsman

### (4) Quest

### (5) Akema

(6) Purac Bioquimica (Massó)

### (7) Chemir

### EXAMPLE 16

### Skin Cream

| Trade Name | Inci Name | % w/w |
|---|---|---|
| Phase A | | |
| ESTOL 3609 | Triethylhexanoin | 5.00 |
| DC 245 Fluid | Cyclopentasiloxane | 2.00 |
| ARLAMOL E | PPG-15 Stearyl Ether | 2.00 |
| Laurex CS | Cetearyl Alcohol | 2.00 |
| Phase B | | |
| PRICERINE 9091 | Glycerine | 2.00 |
| ARLATONE V-175 | Sucrose Palmitate (and) Glyceryl Stearate | 1.10 |
| | (and) Glyceryl Stearate Citrate (and) | |
| | Sucrose (and) Mannan (and) Xanthan | |
| | Gum | |
| Water | Aqua | To 100 |
| Veegum Ultra | Magnesium Aluminium Silicate | 0.10 |
| Yogurtene | yogurt powder | 0.50 |
| Phase C | | 1.00 |
| Phenonip | Phenoxyethanol and Methylparaben and | 1.00 |
| | Ethylparaben and Butylparaben and | |
| | Propylparaben and Isobutylparaben | |
| Soya yogurt Powder | | 0.5 |

### Procedure

Phase A Stir all the ingredient and heat to 80° C. Phase B Add the water in a beacher, add the Yoghurtene Powder, veegum ultra, Pricerine 9091 and arlatone V-175. stir well between each addition. Heat to 80° C. Pour phase A into phase B whilst high shearing.

Cool to 40° C. Add the ingredient of phase C by stirring

### EXAMPLE 17

### Skin Lotion

This non-alcoholic formulation produces a very white, low viscosity emulsion or ‘milk’ packed with rich skin moisturisers and is suitable for spraying from a fine mist pump. Along with its moisturising benefits, it also has excellent fragrance delivery properties.

| Trade Name | Inci Name | % w/w |
|---|---|---|
| SMEC #1 | Aqua & Trimethylolpropane | 50.00 |
| | Tricaprylate/Tricaprate | |
| | & Glycerin& Cetearyl | |
| | Alcohol & Ceteareth-20 & | |
| | Glyceryl Stearate & | |
| | Peg-100 Stearate & Steareth-2 | |
| | & Dimethicone & Ceteth-24 | |
| | & Choleth-24 & | |
| | Phospholipids & Propylene | |
| | Glycol & Methyl | |
| | Paraben & PropylParaben & | |
| | Imidazolidinyl Urea & | |
| | Disodium EDTA | |
| Quest Fragrance | | up to 7.00 |
| Emulgin L (3:1 with | PPG-2-Ceteareth-9 | up to 21.00 |
| fragrance) | | |
| Deionised Water | | to 100.00 |
| Preservative | | as required |
| Naturein Whey Peptide | Hydrolyzed Milk Protein | 0.30 |
| Soya Yogurt powder | | 0.30 |

PREPARATION: Mix the Quest fragrance with the Eumulgin, then add the preservative and SMEC. Separately premix the Whey Peptide with the water. Add water mix to SMEC mix whilst mixing thoroughly, preferably with shear.

### EXAMPLE 18

### Light Yoghurt Hand Cream

A light, everyday emulsion, containing natural soya yogurt powder, a mixture of vitamins and nutrients for skin.

| Trade Name | Inci Name | % w/w |
|---|---|---|
| CARNATION | Mineral Oil | 10.00 |
| CETYL ALCOHOL | | 5.00 |
| CITHROL | GMS (NSE) | 2.50 |
| EUMULGIN B2 | Ceteareth-20 | 2.50 |
| NIPASOL M | Propylparaben | 0.10 |
| NIPAGIN M | Methylparaben | 0.25 |
| DEIONISED WATER | | to 100.00 |
| Soya yogurt Powder | | 0.4 |
| APRICOT EXTRACT | Prunus Armeniaca (Apricot) Extract | 0.50 |

### Method:

In the main vessel, combine the water, Nipagin and soya yoghurt powder, and heat to 80° C. In a side vessel, combine the other ingredients and heat to 75° C. Whilst high shear mixing, add the water phase to the oil phase. Continue mixing until homogenous and then scrape stir cool. Add the fragrance and Apricot Extract, stirring until uniform.

### EXAMPLE 19

### Water in Oil Body Lotion

| | | INCI | % w/w |
|---|---|---|---|
| A. | Arlacel P135 (1) | (PEG-30 Dipolyhydroxystearate) | 1.50 |
| | Monasil PCA (1) | (PCA Dimethicone) | 2.00 |
| | Arlamol HD (1) | (Isohexadecane) | 6.00 |
| | Estasan 3575 (1) | (Caprylic/capric Triglyceride) | 4.00 |
| | Estol 1517 (1) | (Isopropyl Palmitate) | 4.00 |
| | Mineral Oil | (Paraffinum Liquidum) | 2.00 |
| | Sweet Almond Oil | (Prunus dulcis) | 2.00 |
| | Cutina HR (3) | (Hydrogenated Castor Oil) | 0.20 |
| B. | Water up to | (Aqua) | 100.00 |
| | MgSO4•7H2O | (Magnesium Sulfate) | 0.70 |
| | Pricerine 9091 (1) | (Glycerin) | 3.00 |
| | Purasal S/HQ 60 (2) | (Sodium Lactate) | 1.00 |
| | Soya yogurt Powder | | 0.70 |
| C. | Kemaben 2 (4) | (Propylene Glycol, Diazolidinyl | 1.00 |
| | | Urea, Methylparaben, Propylparaben) | |
| D. | TagraPlus A1 (5) | (Retinol Palmitate, Polymethyl | 1.00 |
| | | Methacrylate, Tricaprylin, BHT, | |
| | | Xanthan Gum, Sodium | |
| | | Methylparaben, Chlorophenesin, | |
| | | Aqua) | |
| E. | Perfume | (Parfum) | q.s. |

### Manufacture:

Heat phase A to 80° C. and B to 75° C. Add slowly phase B to A stirring intensively (400 rpm). Homogenise AB for 2 minutes (Silverson: 4.000 rpm). Allow to cool down at 50-45° C. stirring intensively. Add phase D (without homogenisation). Add phases C and E.

Cool down at room temperature keeping high speed.

Viscosity (14° C.): 10.500 mPa·s (Brookfield LVT, Spindle B, 12 rpm)

### EXAMPLE 20

### Body Gel

| Formulation: | | % w/w |
|---|---|---|
| Purified Water | | to 100.00 |
| Soya Yogurt Powder | | 0.30 |
| Denatured Alcohol | Ethanol | 30.00 |
| Sepigel 305 | Polyacrylamide, C13-14 | 3.50 |
| | Isoparaffin & Laureth-7 | |
| Lubrajel TW | Polyglyceryl Methacrylate & | 0.60 |
| | Propylene Glycol | |
| QUEST PERFUME | Parfum | 0.50 |
| Lutrol E 400 | PEG 400 | 0.20 |
| Germaben II-E | Imidazolidinyl Urea | 0.50 |
| | & Methylparaben & Propylparaben | |

### Method:

Combine the water, PEG-400 and Lubrajel TW using a propellor mixer. When homogeneous, add the Sepigel with stirring, until the mixture is thick & uniform. Separately mix the ethanol and perfume. Slowly add this to the water mixture, stirring continuously to ensure complete incorporation. Add the colours and preservative, with stirring.

### EXAMPLE 21

### Loose Face Powder

| Inci Name | % w/w |
|---|---|
| Talc | 74.80 |
| Zinc Stearate | 7.50 |
| Magnesium Carbonate | 1.00 |
| Soya yogurt powder | 0.10 |
| Methylparaben | 0.10 |
| Propylparaben | 0.05 |
| Imidazolidinyl Urea | 0.10 |
| Black Iron Oxide | 0.30 |
| Red Iron Oxide | 3.00 |
| Yellow Iron Oxide | 3.00 |
| Low Lustre Pigment | 10.00 |
| Fragrance | 0.15 |

### EXAMPLE 22

### Formulation for a Blemish Stick

| INCI Name | % w/w |
|---|---|
| Microcrystalline wax Wax | 14.75 |
| Beeswax Wax | 4.50 |
| Propoxylated oleyl alcohol Oil | 50.50 |
| Triclosan Antiseptic | 0.25 |
| Butylated hydroxy anisole Antioxidant | 0.10 |
| Allantoin Skin soothing agent | 0.10 |
| Talc Pigment bulking agent | 9.00 |
| Soya yogurt powder | 0.30 |
| Titanium dioxide Pigment | 10.81 |
| Red iron oxide Pigment | 6.55 |
| Black iron oxide Pigment | 0.30 |
| Yellow iron oxide Pigment | 2.34 |
| Perfume | 0.50 |

### EXAMPLE 23

### Formulation for a Non-Ionic Liquid Foundation

| INCI Name | % w/w |
|---|---|
| Water phase | |
| Deionised water | 79.80 |
| Propylene glycol | 5.00 |
| PEG-20 stearate | 2.00 |
| Methyl paraben | 0.20 |
| Bentone | 1.50 |
| Preservative | |
| Powder phase | |
| Soya yogurt powder | 0.10 |
| Talc Pigment | 5.00 |
| Titanium dioxide | 5.00 |
| Yellow iron oxide | 0.40 |
| Red iron oxide | 0.40 |
| Black iron | 0.10 |
| Fragrance | 0.30 |

### Manufacture

1 Heat water phase to 65° C. and homogenise.
2 Grind powder phase and add to step 1.
3 Homogenise for 30 minutes then add the fragrance below 40° C.

### EXAMPLE 24

### Oil in Water Anti-Acne Skin Treatment Cream

| Ref.: F-01/W-0025/01 | | INCI-Name | % w/w |
|---|---|---|---|
| A. | Brij 721 (1) | (Steareth-21) | 1.50 |
| | Brij 72 (1) | (Steareth-2) | 3.50 |
| | Ecorol 68/30 f (2) | (Cetearyl Alcohol) | 1.50 |
| | Pristerene 9559 (1) | (Stearic Acid) | 2.00 |
| | Arlamol E (1) | (PPG-15 Stearyl Ether) | 8.00 |
| | DC 200/100 (3) | (Dimethicone) | 0.30 |
| B. | Water up to | (Aqua) | 100.00 |
| | Structure Zea (4) | (Hydroxypropyl Starch Phosphate) | 3.00 |
| | Atlas G-2330 (1) | (Sorbeth-30) | 4.00 |
| | Soya yogurt Powder | | 0.20 |
| C. | Azeloglicina (7) | (Potassium Azelaoyl Diglycinate) | 3.00 |
| D. | Arlasolve DMI (1) | (Dimethyl Isosorbide) | 5.00 |
| | Salicylic Acid | (Salicylic Acid) | 1.00 |
| E. | Kemaben 2 (5) | (Propylene Glycol, Diazolidinyl | 1.00 |
| | | Urea, Methylparaben, Propylparaben) | |
| F. | Perfume L.F. | (Parfum) | q.s. |
| | 9115/8 (6) | | |

### Manufacture:

Disperse Structure Zea in water (room temperature). Add the rest of ingredients of phase B. Heat A and B to 75° C. Add A to B whilst stirring slowly. Homogenise AB for 1½ minutes (Silverson: 3.000 rpm). Allow to cool down at 45-40° C. whilst stirring slowly. Add phases C, D, E and perfume one by one. Cool down to room temperature whilst stirring slowly.

Viscosity (18° C.): 67.900 mPa·s (Brookfield LVT, Spindle F. 12 rpm)
pH=5.20 (NaOH, 20%)

### EXAMPLE 25

### Body Refining Gel (Anti-Cellulite)

| Formulation: | | % w/w |
|---|---|---|
| Purified Water | | to 100.00 |
| Soya Yogurt Powder | | 0.30 |
| Denatured Alcohol | Ethanol | 30.00 |
| Sepigel 305 | Polyacrylamide, C13-14 Isoparaffin & Laureth-7 | 3.50 |
| QUESTICE PLUS | Menthyl PCA & Menthol & DPG | 2.00 |
| Lubrajel TW | Polyglyceryl Methacrylate & Propylene Glycol | 0.60 |
| QUEST PERFUME | Parfum | 0.50 |
| Lutrol E 400 | PEG 400 | 0.20 |
| ACTISCULPT ™ | Dipropylene glycol and Commiphora Mukul 1.00 | 0.50 |
| | Resin Extract and Coleus Forskohlii Extract Germaben II- | |
| | E Imidazolidinyl Urea & Methylparaben & Propylparaben | |
| DYE (blue no 1 0.5%) | Cl42090 | qsp |

### Method:

Combine the water, PEG400 and Lubrajel TW using a propellor mixer. When homogeneous, add the Sepigel with stirring, until the mixture is thick & uniform. Separately mix the ethanol, Questice Plus, Actisculpt and perfume. Slowly add this to the water mixture, stirring continuously to ensure complete incorporation. Add the colours and preservative, with stirring.

### EXAMPLE 26

### Facial Firming Night Cream with Soya Yoghurt

| | | |
|---|---|---|
| Trade Name | INCI Name | % w/w |

Phase A

| | | |
|---|---|---|
| Mineral Oil | Mineral Oil | 7.50 |
| Arlamol HD | Isohexadecane | 7.50 |
| Prisorine 3700 | Polyglyceryl-3-diisosotearate | 5.00 |
| Estol 3609 | Trihexanoin | 4.00 |
| Soya Yoghurt Powder | | 0.50 |
| Ceramide II | Ceramide 2 | 0.10 |

Phase B

| | | |
|---|---|---|
| Water | Aqua | To 100.00 |
| Princerine 9091 | Glycerin | 5.00 |
| MgSO₄—7 H₂O | Magnesium sulfate | 1.00 |
| NaCl | Sodium chloride | 1.00 |

Phase C

| | |
|---|---|
| Preservative | qsp |
| Quest Fragrance | qsp |

PREPARATION: Heat Phase A and B separately to 75-80° C. Slowly, add phase B to A mixing with rapid stirring. Add phase C and homonogenise ABC for 2 minutes. Allow to cool down to room temperature with stirring. Add perfume and preservative as required.

### EXAMPLE 27

### High Protection Sunscreen Lotion

This thick, creamy lotion is easily absorbed into the skin to give a smooth, non-greasy feel. It contains a combination of sunscreen agents which should give a high Sun Protection Factor in the region of 15.

Pongamol is a natural UV filter. Soya Yogurt Powder help the moisturisation of the skin.

### Formulation:

| Trade Name | INCI Name | % w/w |
|---|---|---|
| Phase A | | |
| Dow corning 344. | Cyclomethicone | 10.00 |
| Escalol 50 | 2-ethylhexyl-4-dimethyl-3-amino | 4.00 |
| | benzoate | |
| Escalol 557 | Octyl methoxycinnamate | 4.00 |
| Parsol MCX | (Ethylhexyl Methoxycinnamate) | 2.00 |
| GMS Non SE | | 1.50 |
| Stearic acid triple | | 3.00 |
| pressed | | |
| Generol 122N | Soya sterol | 0.40 |
| Dow Corning 1401 | | 6.00 |
| Pongamol | | 0.15 |
| Phase B | | |
| Water up to | (Aqua) | 100.00 |
| Sorbitol (70%) | | 5.00 |
| TEA | | 0.90 |
| Soya yogurt powder | | 0.30 |
| Preservative | | qsp |
| Phase C | | |
| fragrance | Parfum | q.s. |

### Manufacture:

Mix the 344 fluid and Q2-1401 fluid of phase A together and heat to 75° C. Mix the remaining ingredients of phase A together and heat to 75° C. Heat phase B to 75° C.

Add the 344 and Q2-1401 fluids to the rest of phase A with high speed stirring, then add phase B. Stir until cool, avoiding aeration.

### EXAMPLE 28

### Aftersun Creme Gelee

| Trade Name | INCI Name | % w/w |
|---|---|---|
| Phase A | | To 100 |
| Deionised Water | | |
| Klucel 99-H | Hydroxypropylcellulose | 0.20 |
| Carbopol Ultrez 10MV | Carbomer | 0.80 |
| Soya yogurt powder | | 0.30 |
| Phase B | | |
| SOOTHEX | Boswellia Serrata Extract | 1.50 |
| Questice Liquid | Menthyl PCA | 1.50 |
| Euxyl K400 | Methyldibromoglutaronitrile and | 0.10 |
| | Phenoxyethanol | |
| Cremophor RH 40 | PEG-40 hydrog.castor oil | 1.30 |
| Phase C: | | |
| Neutrol TE | Tetrahydroxypropyl-ethylenediamine | 0.20 |
| Deionised water | | 4.50 |
| Quest perfume | | qs |

Deionised water and Klucel were combined under high shear was used for 1 hour, until the mixture was uniform and lump free. Carbomer was added and left to wet out. Phase B was combined with gentle heating and added to A with shear (but not too high in order to avoid breaking down the structure). Phase C was combined and added with a propeller stirrer to ensure thorough mixing.

### EXAMPLE 29

### Physical Block Sunscreen Stick

| Trade Name | INCI Name | % w/w |
|---|---|---|
| Phase A | | |
| Propylene glycol | | 10.00 |
| Promyristyl pm3 | PPG-3 myristyl ether | 21.00 |
| Procetyl aws | | 7.00 |
| Sodium stearate | | 6.00 |
| Phase B | | |
| Dow corning 345 fluid | | 10.50 |
| Dow corning 3225c fluid | | 10.50 |
| Purified water | | to 100.00 |
| Tioveil ipm | | 12.50 |
| Soya Yogurt Powder | | 0.50 |
| Phase C | | |
| QUEST FRAGRANCE | | as required |

PREPARATION: Combine the ingredients of phase A and heat, with stirring, to 85° C. Allow to cool to 60° C. before adding each of the ingredients of phase B. Add the fragrance and mix until homogeneous. Fill into containers and allow to cool.

### EXAMPLE 30

### Two-Phase “Satin” Cologne

This product is designed to be applied to the skin, to leave a long-lasting, subtle veil of fragrance. It also leaves the skin feeling smooth and soft. The product will separate into two phases on standing, and should be shaken well before use.

| FORMULATION: | % w/w |
|---|---|
| PHASE A: | |
| ETHANOL | 40.00 |
| QUEST FRAGRANCE | as required |
| PHASE B: | |
| PURIFIED WATER | to 100.00 |
| ORGASOL 2002D NAT COS | 10.00 |
| d-PANTHENOL 75L | 1.20 |
| Soya Yogurt powder | 0.50 |

PREPARATION: Combine the ingredients of phase A. Separately combine the ingredients of phase B, before adding to phase A with rapid stirring. Package immediately.

### EXAMPLE 31

### Roll-on Fragrance

| FORMULATION: | % w/w |
|---|---|
| Ethanol, denatured | 40.00 |
| NATROSOL 250 HR (i) | 0.60 |
| PURIFIED WATER | to 100.00 |
| Soya Yogurt Powder | 0.10 |
| QUEST FRAGRANCE | 15.00 |

(i) Hydroxyethylcellulose

PREPARATION: Heat the water to 70-75° C., add the Natrosol using high speed mixer and stir well until homogeneous. Allow to cool. Add the Natrosol solution to the alcohol and mix well. Add the fragrance and mix well.

### EXAMPLE 32

### Dry Fragrance Spray

This product was developed for use where a very dry application is required.

| FORMULATION: | % w/w |
|---|---|
| CONCENTRATE: | |
| BENTONE GEL IPP (or IPM) (i) | 13.65 |
| SILICONE FLUID DC 344 (ii) | to 100.00 |
| LO-MICRON TALC | 0.5 |
| Soya Yogurt Powder | 0.40 |
| METHYLAL | 20.00 |
| QUEST FRAGRANCE | up to 50.00 |
| AEROSOL FILL: | |
| CONCENTRATE | 40.00 |
| HYDROCARBON PROPELLANT | 60.00 |
| 40 psig | |

(i) Isopropyl palmitate (and) stearalkonium hectorite (and) propylene carbonate
(ii) Cyclomethicone

PREPARATION: Thoroughly blend the Bentone Gel with the volatile silicone. Mix in the Lo-Micron Talc and fragrance with rapid stirring. Add the Methylal and mix well.

### EXAMPLE 33

### Roll-On Antiperspirant/Deodorant (Emulsion Tape)

This opaque lotion, contains no alcohol and so is mild on the skin. It is suitable for use on sensitive or freshly shaved skin.

### Formulation:

| Trade Name | INCI Name | % w/w |
|---|---|---|
| Phase A | | To 100 |
| Deionised Water | | |
| Veegum r | Magnesium aluminium silicate. | 1.00 |
| Soya Yogurt Powder | | 0.30 |
| PHASE B | | 8.00 |
| Arlacel 165 | | |
| Phase C | | |
| | Aluminium chlorhydrate (50% solution) | 55.00 |
| Phase D | DOW CORNING 344 FLUID | 7.00 |
| Quest fragrance | | as |
| | | required |

PREPARATION: Add the Veegum to the water slowly with constant agitation until smooth. Heat to 70° C. Separately heat the Arlacel to 75° C., and add to phase A. Stir whilst cooling to 50° C. and add to the emulsion. Stir thoroughly until cool. Finally add phase D.

### EXAMPLE 34

### Apricot Yoghurt Soap

### Formulation:

| Prisavon 9222 | Vegetable Soap Base | to 100.00 |
|---|---|---|
| APRICOT EXTRACT | Prunus Armeniaca (Apricot) Extract | 0.50 |
| Soya yogurt powder | | 0.50 |
| Quest Fragrance | Parfum | 0.50 |

### Method:

Mill the Prisavon once and then add the remaining ingredients. Mill a further three times, before passing through a vacuum plodder. Finally, stamp into bars

### EXAMPLE 35

### Liquid Soap

### Formulation:

| | | % w/w |
|---|---|---|
| Deionised water | | to 100.00 |
| Empicol esb3 | Sodium Laureth Sulfate (28%) | 30.00 |
| Tego betain f50 | Cocamidopropyl Betaine | 10.00 |
| Glycerine | | 4.00 |
| Euperlan pk771 | Glycol Distearate & Sodium Laureth | 3.00 |
| Sulfate & Cocamide | MEA & Laureth-10 | |
| Arlatone sci | Sodium Cocoyl Isethionate | 2.00 |
| Antil 171 | PEG-18 Glceryl Oleate/Cocoate | 1.00 |
| Sodium chloride | | 1.00 |
| Isolan go33 | Polyglyceryl-3 Oleate | 1.00 |
| Soya Yogurt | | 1.00 |
| Lactil | Sodium Lactate & Sodium PCA & | 1.00 |
| | Hydrolysed Animal Protein & Fructose | |
| | & Urea & Niacinamide & Inositol & | |
| | Sodium Benzoate & Lactic Acid | |
| Citric acid | | 0.12 |
| Nervanaid ba2 | Disodium EDTA | 0.10 |
| Kathon cg | Methylchloroisothiazolinone & | 0.06 |
| | Methylisothiazolinone | |
| Quest perfume | | q.s. |

### Method:

Dissolve the EDTA in water, stirring to dissolve. Add the Arlatone SCI, warming gently to dissolve. Separately mix the Questamide H with the Empicol, then add the Lactil, Glycerine, Citric Acid, Isolan, Betain and Antil. Warm and stir until thick and homogeneous: Slowly add the water phase with gentle stirring. Cool to room temperature, then add the remaining ingredients to the main batch, stirring until homogeneous between additions.

1. (canceled)

2. (canceled)

3. A method of providing conditioning benefits to skin or hair, comprising topical application to skin or hair of a fermentation product of soya bean extract.

4. A composition for treatment or care of skin, comprising a fermentation product of soya bean extract.

5. A composition according to claim 4, not including vitamin C or a derivative thereof.

6. A composition for treatment or care of hair, comprising a fermentation product of soya bean extract.

7. A method of making a composition for care or treatment of skin or hair, comprising including as an ingredient a fermentation product of a soya bean extract in dried condition.

8. The method of claim 3 or claim 7, wherein the soya bean extract comprises soya milk.

9. The method of claim 3 or claim 7, wherein the fermentation product is a product of lactic acid fermentation.

10. The method of claim 8, wherein the fermentation product is obtained by fermentation with a lactobacillus species and a Streptococcus species.

11. The method of claim 3 or claim 7, wherein the fermentation product is soya yoghurt.

12. The method of claim 7, wherein the fermentation product is in dried condition for inclusion in said composition.

13. The method of claim 12, wherein the dried fermentation product includes one or more preservatives.

14. The method of claim 12 or 13, wherein the dried fermentation product has a sugar content of less than 10% by weight.

15. The method of claim 12, wherein the dried fermentation product has a lipids content of less than 10% by weight.

16. The method of claim 12, which comprises from 5 to 75% by weight of flow aid.

17. The method of claim 3 or claim 7, which includes added soy proteins and/or hydrolysed soy proteins in an amount of from 2 to 25% by weight.

18. A dried fermentation product of soya bean extract with a lipids content of less than 10% by weight.

19. The dried fermentation product of claim 18, wherein the lipids content of less than 5% by weight.

20. The dried fermentation product of claim 18, wherein the lipids content of less than 3% by weight.

21. A composition according to claim 4 or 6, wherein the soya bean extract comprises soya milk.

22. A composition according to claim 4 or 6, wherein the fermentation product is a product of lactic acid fermentation.

23. A composition according to claim 22, wherein the fermentation product is obtained by fermentation with a lactobacillus species and a Streptococcus species.

24. A composition according to claim 22, wherein the fermentation product is soya yoghurt.

25. A composition according to claim 22, wherein the fermentation product is added to said composition in the dried condition.

26. A composition according to claim 25, wherein the dried fermentation product includes one or more preservatives.

27. A composition according to claim 25, wherein the dried fermentation product has a sugar content of less than 10% by weight.

28. A composition according to claim 25, wherein the dried fermentation product has a lipid content of less than 10% by weight.

29. A composition according to claim 4 or 6, which comprises from 5 to 75% by weight of flow aid.

30. A composition according to claim 4 or claim 6, which includes from 2 to 25% by weight of soy proteins and/or hydrolysed soy proteins.
